(19) United States
(12) Patent Application Publication
(10) Pub. No.: US 2026/0127914 A1
(43) Pub. Date: May 7, 2026

(54) APPARATUS AND METHOD FOR RECOGNIZING STEREOTYPED ACTIONS BASED ON ARTIFICIAL INTELLIGENCE

(71) Applicant: ELECTRONICS AND TELECOMMUNICATIONS RESEARCH INSTITUTE, Daejeon (KR)

(72) Inventors: Cheolhwan Yoo, Daejeon (KR); Jang-Hee Yoo, Daejeon (KR); JAEYOON Jang, Daejeon (KR)

(21) Appl. No.: 19/355,463

(22) Filed: Oct. 10, 2025

(30) Foreign Application Priority Data
Nov. 7, 2024 (KR) 10-2024-0157102

(51) Int. Cl. G06V 40/16; G06V 10/25; G06V 10/74; G06V 10/764; G06V 10/82; G06V 20/40; G06V 20/70; G16H 50/20

(52) U.S. Cl. CPC G06V 40/174; G06V 10/25; G06V 10/761; G06V 10/764; G06V 10/82; G06V 20/41; G06V 20/70; G06V 40/161; G06V 40/168; G16H 50/20

(57) ABSTRACT

Provided is an apparatus for recognizing a stereotyped action, which includes: a memory storing a learning video dataset including a stereotyped action of a designated disabled child; and a processor functionally connected to the memory, wherein the processor includes: a text encoder configured to extract first features from a composite description phrase related to a facial expression and an action of the child included in the learning video dataset; a video encoder configured to output second features related to a facial expression and an action of the child from the learning video dataset; and a contrastive learning unit configured to learn a similarity between the first and second features that are paired with each other among the first features and the second features to model the video encoder.

### CROSS-REFERENCE TO RELATED APPLICATION

This application claims priority to and the benefit of Korean Patent Application No. 10-2024-0157102, filed on Nov. 7, 2024, the disclosure of which is incorporated herein by reference in its entirety.

### BACKGROUND

### 1. Field of the Invention

Various embodiments disclosed in this document relate to a technology for recognizing a user action.

### 2. Description of Related Art

According to the U.S. Centers for Disease Control and Prevention (CDC), the prevalence of autism spectrum disorder (ASD) in children has been steadily increasing every year, from 1 in 54 in 2016 to 1 in 36 in 2020. In Korea, as well, the prevalence is high, with 1 in 38 (2.64%) affected and a significant increase (an average annual growth of 6.6%).

Early diagnosis of ASD children is very important in terms of enabling treatment within the critical window and preventing secondary neurological damage and accumulation of actional problems to some extent. However, conventional diagnostic systems have mainly relied on labor-intensive and repetitive tests performed by medical professionals. Therefore, the approach took much time and often resulted in missing the critical window for early diagnosis, which is very important for the prognosis of ASD children.

To resolve these issues, technologies that support ASD diagnosis by analyzing stereotyped actions, which are the main actional indicators of ASD children, using artificial intelligence (AI)-based automated analysis devices are being widely studied. These studies have attracted significant interest from researchers and clinicians.

### SUMMARY OF THE INVENTION

Conventional AI-based stereotyped action recognition and detection methods may have several limitations as follows. For example, action recognition technologies may recognize the final action class from a specific pattern analyzed from video data using a black-box AI model. However, such black-box models not only have low interpretability, but also have difficulty providing an intermediate reasoning leading to an inference result. Therefore, it is difficult for medical professionals to trust, accept, and clinically utilize AI diagnoses without a basis for recognizing stereotyped actions. As another example, conventional AI-based stereotyped action recognition technologies simply analyze only physical movements and actional patterns of children, making it difficult to understand and interpret the composite actional characteristics of ASD children. The actions of ASD children are closely related to their emotional states, and the same action may have different meanings and interpretations depending on the child's emotional state.

Various embodiments disclosed in this document may provide an apparatus and method for recognizing stereotyped actions based on artificial intelligence with which it is possible to assist in the diagnosis of children with autism spectrum disorder by analyzing video data.

According to an aspect of the present invention, there is provided an apparatus for recognizing a stereotyped action, which includes: a memory storing a learning video dataset including a stereotyped action of a designated disabled child; and a processor functionally connected to the memory, wherein the processor includes: a text encoder configured to extract first features from a composite description phrase related to a facial expression and an action of the child included in the learning video dataset; a video encoder configured to output second features related to a facial expression and an action of the child from the learning video dataset; and a contrastive learning unit configured to learn a similarity between the first and second features that are paired with each other among the first features and the second features to model the video encoder.

According to an aspect of the present invention, there is provided an apparatus for recognizing a stereotyped action recognition device, which includes: a memory storing first features of a list of composite description phrases describing a stereotyped action of a designated disabled child in relation to a facial expression; and a processor functionally connected to the memory, wherein the processor includes: a video encoder configured to extract second features related to an action and a facial expression of a subject to be diagnosed from one piece of video data; and an action recognition unit configured to infer a type of the action included in the one piece of video data based on the similarity between the first features and the second features.

According to an aspect of the present invention, there is provided a method of recognizing a stereotyped action, which is performed by at least one processor, which includes: encoding at least one composite descriptive phrase related to a learning video dataset to extract first features; encoding the learning video dataset using a video encoder to output second features related to a facial expression and an action of a subject to be diagnosed; and learning a similarity between the first and second features that are paired with each other among the first features and the second features to model the video encoder such that the similarity between the first and second features paired with each other increases.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram illustrating a computing system for providing a method of recognizing a stereotyped action according to an embodiment;

FIG. 2 is a block diagram illustrating an apparatus for recognizing a stereotyped action in a learning stage according to an embodiment;

FIGS. 3 and 4 are block diagrams illustrating an input and an output of an apparatus for recognizing a stereotyped action in a learning stage according to an embodiment;

FIGS. 5 and 6 are block diagrams illustrating an apparatus for recognizing a stereotyped action in an inference stage according to an embodiment;

FIG. 7 illustrates an example of intermediate concept information according to an embodiment;

FIG. 8 is a flowchart showing a method of learning stereotyped action recognition according to an embodiment; and

FIG. 9 is a flowchart showing a method of recognizing a stereotyped action according to an embodiment.

In relation to the description of the drawings, identical or similar reference numerals may be used for identical or similar components.

### DETAILED DESCRIPTION OF EXEMPLARY EMBODIMENTS

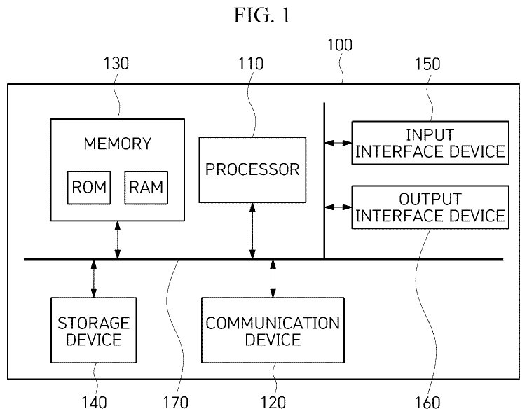

FIG. 1 is a block diagram illustrating a computing system for providing a method of recognizing a stereotyped action according to an embodiment.

Referring to FIG. 1, an apparatus 100 for recognizing a stereotyped action according to the embodiment may be an apparatus for recognizing a stereotyped action based on composite information integration for supporting autism spectrum disorder (ASD) diagnosis. Alternatively, the method of recognizing a stereotyped action according to the embodiment may be implemented in a computer system such as a computer-readable recording medium.

The apparatus 100 for recognizing a stereotyped action according to the embodiment may include at least one of a processor 110, a memory 130, an input interface device 150, an output interface device 160, and a storage device 140 that perform communication through a bus 170.

The apparatus 100 for recognizing a stereotyped action may further include a communication device 120 coupled to a network. The communication device 120 may transmit and receive data (e.g., video data) to and from an external electronic device (e.g., a camera module, a user terminal).

The input interface device 150 may obtain or receive a user input related to a request for recognizing a stereotyped action. The output interface device 160 may include, for example, a display and may visually output a result of the recognition (inference results and intermediate concepts) of the stereotyped action.

The memory 130 and the storage device 140 may include various forms of volatile or nonvolatile storage media. For example, the memory 130 may include a read only memory (ROM) or a random access memory (RAM). In an embodiment of the present disclosure, the memory 130 may be located inside or outside the processor 110 and may be connected to the processor 110 through various known means. The memory 130 may store various forms of data used by at least one component of the apparatus 100 for recognizing a stereotyped action (e.g., the processor 110). The data may include, for example, input data or output data for software and instructions related thereto. For example, the memory 130 may store at least one instruction and data for recognizing a stereotyped action.

The processor 110 may be a central processing unit (CPU), or a semiconductor device that executes instructions stored in the memory 130 or the storage device 140. According to an embodiment, the processor 110 may include: a text encoder 115 configured to extract first features from at least one composite description phrase related to a facial expression and an action included in a learning video dataset; a video encoder 116 configured to output second features related to the facial expression and the action by encoding the learning video dataset; and a contrastive learning unit 117 configured to learn a similarity between first and second features that are paired with each other among the first features and the second features to model the video encoder 116. Hereinafter, a detailed description will be provided.

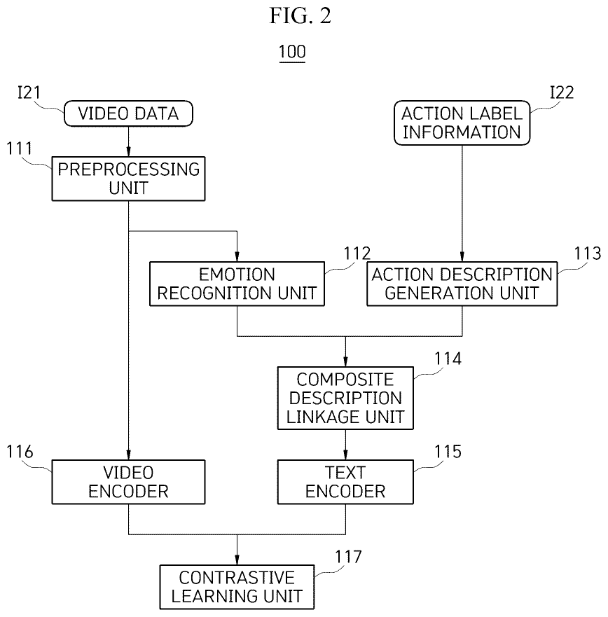

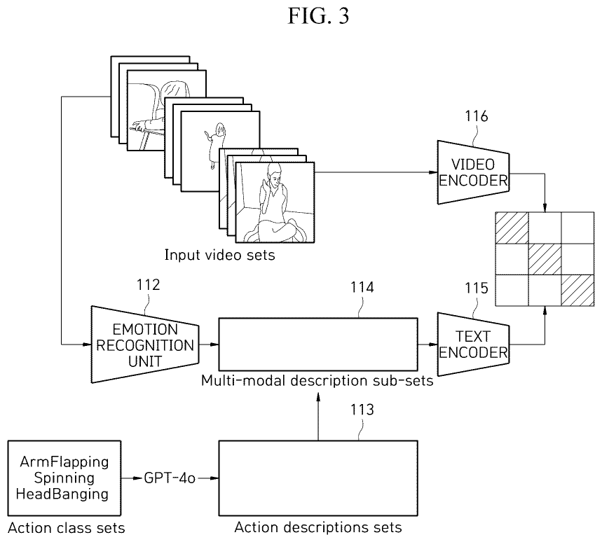

FIG. 2 is a block diagram illustrating an apparatus for recognizing a stereotyped action in a learning stage according to an embodiment, and FIGS. 3 and 4 are block diagrams illustrating an input and an output of an apparatus for recognizing a stereotyped action in a learning stage according to an embodiment.

Referring to FIG. 2, an apparatus 100 for recognizing a stereotyped action according to the embodiment may include a text encoder 115, a video encoder 116, and a contrastive learning unit 117. In an embodiment, in the apparatus 100 for recognizing a stereotyped action, some components may be omitted or additional components may be added. For example, the apparatus 100 for recognizing a stereotyped action may further include an emotion recognition unit 112, an action description generation unit 113, and a composite description linkage unit 114. The apparatus 100 for recognizing a stereotyped action may further include a preprocessing unit 111. In addition, some of the components of the apparatus 100 for recognizing a stereotyped action may be combined into a single component but may perform the functions of the components before the combination. For example, at least one component among the preprocessing unit 111, the emotion recognition unit 112, the action description generation unit 113, the composite description linkage unit 114, the text encoder 115, the video encoder 116, and the contrastive learning unit 117 may be combined or omitted.

According to an embodiment, the preprocessing unit 111, the emotion recognition unit 112, the action description generation unit 113, the composite description linkage unit 114, the text encoder 115, the video encoder 116, and the contrastive learning unit 117 may be a software module or a hardware module included in the processor 110 of the apparatus 100 for recognizing a stereotyped action, or executed by the processor 110 of the apparatus 100 for recognizing a stereotyped action.

According to an embodiment, the emotion recognition unit 112 may determine an emotion word corresponding to an emotion of an autism spectrum disorder (ASD) child from a learning dataset.

For example, the emotion recognition unit 112 may, when video data of a learning video dataset is input one by one, classify the child's emotion based on a facial expression feature of a face region included in the video data using an emotion classification model. The emotion recognition unit 112 may output an emotion word corresponding to the classified emotion (an emotion word matched with the emotion). The learning video dataset is video data in which actions of a child with ASD are recorded, and may be obtained from, for example, the memory 130.

In an embodiment, the emotion recognition unit 112 may classify various types of recognized emotions according to an emotion model. For example, the emotion recognition unit 112 may classify the types of emotions into seven emotions of happiness, sadness, disgust, anger, surprise, fear, and neutrality according to a categorical model.

According to an embodiment, the action description generation unit 113 may generate action description phrases describing a stereotyped action based on label information of the stereotyped action. The label information of the stereotyped action may be, for example, a stereotyped action label (e.g., a name) of a child with ASD, such as “arm flapping”, “headbanging”, and “spinning”. For example, the action description generation unit 113 may generate a plurality of action description phrases for each piece of label information of the stereotyped action. The action description generation unit 113 may instruct, for example, a large language model (e.g., GPT-4o) to generate ten action description phrases (texts) for each class (type) of stereotyped actions, focusing on the temporal and spatial aspects of the actions, and may obtain the ten action description phrases as a response from the large language model.

The following phrases represent examples of some of the ten action description phrases that are finally selected by reviewers (e.g., medical professionals) from among the class-specific action description phrases obtained from the large language model.

[Example of action description phrase]
-------------------------------------------------------
- “A video of arm flapping.”
- “Repeatedly moving arms up and down in quick succession.”
- “Flapping arms in a rapid, rhythmic motion.”
- “Continuous up-and-down arm motion.”
- “Hands positioned near shoulder height during flapping.”
- “A video of spinning”
- “Continuous turning in a circular motion”
- “Repeatedly spinning in place without stopping.”
- “Rapid rotation around a fixed point.”
- “Movement occurring in a horizontal circular plane.”
- “A video of headbanging”
- “Repeatedly hitting head against a surface in a rhythmic manner.”
- “Continuous head banging occurring at regular intervals.”
- “Rapid, repetitive head movements hitting a surface.”
- “Head making contact with a hard surface repeatedly.”
-------------------------------------------------------

According to an embodiment, the composite description linkage unit 114 may generate a composite description phrase using the emotion words and the action description phrases. For example, the composite description linkage unit 114 may obtain the emotion words and the plurality of action description phrases from the emotion recognition unit 112 and the action description generation unit 113, respectively. The composite description linkage unit 114 may randomly select a single action description phrase from the plurality of action description phrases. The composite description linkage unit 114 may generate a composite description phrase by combining the emotion word and the selected action description phrase.

For example, a child included in the video data may show an agitated facial expression or emotion and exhibit a stereotyped action corresponding to “headbanging.” In this case, the composite description linkage unit 114 may generate a composite description phrase such as “Repetitive head movements occurring at regular intervals with a feeling of fear” by combining the emotion state and one of the action description phrases. For another example, when a child in the video data shows a stereotyped action of rapidly and repeatedly moving his or her arms up and down with a happy facial expression, the composite description linkage unit 114 may generate the first composite description phrase below. Alternatively, when a child in the video data shows a stereotyped action of continuously rotating in a circle without a facial expression, the composite description linkage unit 114 may generate the second composite description phrase below. Alternatively, when a child in the video data shows a stereotyped action of moving his or her head regularly and repeatedly in fear, the composite description linkage unit 114 may generate the third composite description phrase below.

Repeatedly moving arms up and down with a feeling of happiness
Continuous turning in a circular motion with a feeling of neutral
Repetitive head movements occurring at
regular intervals with a feeling of fear

According to an embodiment, the text encoder 115 may extract first features by encoding at least one composite description phrase related to facial expressions and actions included in the learning video dataset. For example, when the text encoder 115 obtains the composite description phrase from the composite description linkage unit 114, the text encoder 115 may tokenize the composite description phrase and convert the tokenized composite description phrase into a text embedding (a vector).

In an embodiment, the text encoder 115 may use a transformer-based model. For example, the text encoder 115 may be a text encoder of a pre-trained contrastive language-image pretraining (CLIP) network.

According to an embodiment, the video encoder 116 may output second features related to facial expressions and actions by encoding the learning video dataset.

In an embodiment, the video encoder 116 may be implemented with a 3D convolutional neural network (CNN) or video transformer-based structure to efficiently utilize temporal information of time series data. For example, the video encoder 116 may be implemented with a Video Swin Transformer network according to performance and complexity conditions and may extract second features related to actions and expressions of a child included in the video data.

According to an embodiment, the contrastive learning unit 117 may model the video encoder 116 by learning the similarity between the first and second features that are paired with each other among the first features and the second features.

For example, the learning video dataset may include a plurality of pieces of learning video data each including a different stereotyped action of an ASD child. In this case, the video encoder 116 and the text encoder 115 may output first features and second features that may be distinguishable (e.g., class-separated) for each of the plurality of stereotyped actions. Accordingly, the contrastive learning unit 117 may adjust the weights of the video encoder 116 such that the feature similarity (e.g., cosine similarity) between the first and second features that form the same pair among the first and second features related to the plurality of stereotyped actions is maximized and the feature similarity between the first and second features that form different pairs is minimized. In other words, the contrastive learning unit 117 may perform learning such that the feature similarity of a pair of first and second features corresponding to a composite description phrase and video data of the same stereotyped action within an input batch of the video encoder 116 is maximized, and the similarity with the other pairs in the batch is minimized.

According to various embodiments, the apparatus 100 for recognizing a stereotyped action may further include a preprocessing unit 111. The preprocessing unit 111 may detect a face region from each frame of the learning video dataset and provide the detected face region to the video encoder 116 and the emotion recognition unit 112.

According to various embodiments, the apparatus 100 for recognizing a stereotyped action may be used to classify stereotyped actions related to diseases other than ASD. In this case, the learning video dataset, the action description phrases related to the stereotyped actions and composite description phrases may be prepared differently.

Through the above-described learning process, the video encoder 116 may be modeled (or configured) to more accurately detect second features related to facial expressions and actions of ASD children. Thereafter, the video encoder 116 may be used for ASD diagnosis based on facial expressions and actions of a user.

As described above, the apparatus 100 for recognizing a stereotyped action according to an embodiment may generate composite description phrases describing facial expressions and actions to automatically recognize and analyze stereotyped actions frequently appearing in ASD children based on AI to support medical professionals in diagnosing ASD children.

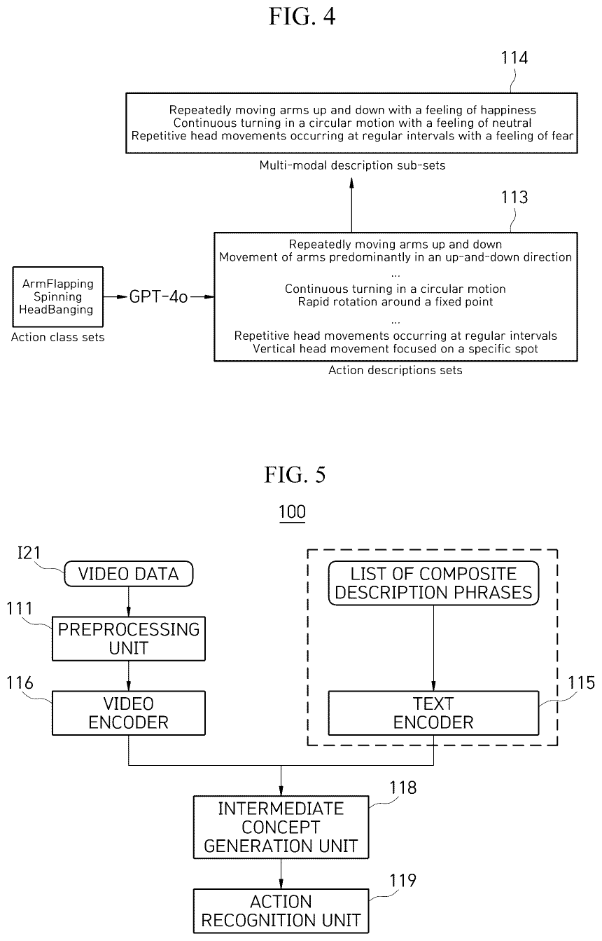

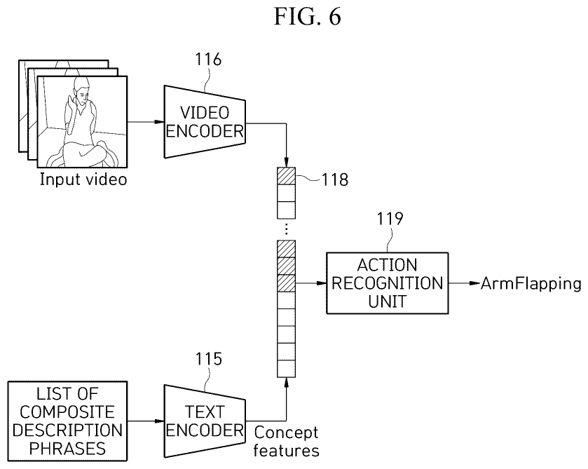

FIGS. 5 and 6 are block diagrams illustrating an apparatus for recognizing a stereotyped action in an inference stage according to an embodiment.

Referring to FIGS. 5 and 6, an apparatus 100 for recognizing a stereotyped action according to an embodiment (e.g., a processor 110 of FIG. 1) may include a text encoder 115, a video encoder 116, an intermediate concept generation unit 118, and an action recognition unit 119. In an embodiment, in the apparatus 100 for recognizing a stereotyped action, some components may be omitted or additional components may be added. For example, the text encoder 115 may be omitted. In addition, some components of the apparatus 100 for recognizing a stereotyped action may be combined into a single component but may perform the functions of the components before the combination. For example, at least one component among the text encoder 115, the video encoder 116, the intermediate concept generation unit 118, and the action recognition unit 119 may be combined or omitted. According to an embodiment, the text encoder 115, the video encoder 116, the intermediate concept generation unit 118, and the action recognition unit 119 may be a software module or a hardware module included in the processor 110 of the apparatus 100 for recognizing a stereotyped action, or executed by the processor 110 of the apparatus 100 for recognizing a stereotyped action.

The text encoder 115 may extract first features by encoding a list of composite description phrases related to facial expressions and actions of an ASD child. The list of composite description phrases may be configured in the form of a concatenation of a list of 30 description phrases for stereotyped actions utilized in pre-learning and a list of 7 emotion words. For example, the list of composite description phrases may be configured as in the following example.

A feeling of happiness
A feeling of neutral
A feeling of fear
...
Repeatedly moving arms up and down in quick succession
Movement of arms predominantly in an up-and-down direction
...
Continuous turning in a circular motion
Rapid rotation around a fixed point
...
Rapid, repetitive head movements occurring at regular intervals
Vertical head movement focused on a specific spot

The video encoder 116 may extract second features related to actions and expressions of a user by encoding one piece of video data.

The intermediate concept generation unit 118 may produce a similarity (a similarity vector) between the first features related to the list of composite description phrases and the second features related to the one piece of video data. The similarity vector may include a value indicating the degree to which the input video and the list of composite description phrases match. The similarity vector may be used as an input for the final action classification of the action recognition unit 119 through a fully connected layer.

The intermediate concept generation unit 118 may generate similarity-related information (or inference basis information) between the list of composite description phrases and an action and a facial expression in the one piece of video data. The similarity-related information may include text or diagrams (tables, graphs) indicating the degree to which the input video and the list of composite description phrases match. Accordingly, the intermediate concept generation unit 118 according to an embodiment may predict not only the action class in the process of recognizing the stereotyped action of a child with autism, but also provide a more sophisticated and interpretable analysis by combining a specific description of the action and an emotional state.

The action recognition unit 119 may infer the type of the action included in the one piece of video data based on the similarity vector between the first features and the second features. The action recognition unit 119 may obtain the similarity vector from the intermediate concept generation unit 118 through a fully connected layer. The action recognition unit 119 may output the inference result (the type of the stereotyped action) (e.g., headbanging) corresponding to the video data.

According to various embodiments, the list of composite description phrases may be consistently used in the inference stage. In this case, the text encoder 115 of the apparatus 100 for recognizing a stereotyped action may be omitted. In this case, the first features corresponding to the list of composite description phrases may be obtained in advance through encoding using the text encoder 115 and stored in the memory 130. Thereafter, the intermediate concept generation unit 118 may obtain the first features from the memory 130 and calculate the similarity between the obtained first features and the second features extracted from the facial expressions and actions corresponding to one piece of video data.

According to various embodiments, the intermediate concept generation unit 118 and the action recognition unit 119 may output (e.g., display) the similarity-related information and the result of the inference through the output interface device 160.

As described above, the apparatus 100 for recognizing a stereotyped action according to an embodiment not only provides an automatic diagnosis of ASD based on emotions and actions included in a child's action recording video, but also provides decision-making process information (intermediate concept information or similarity-related information) that combines a detailed description of the action and composite information on the emotional state associated with the action, thereby supporting an expert to interpret and verify the decision-making of the AI and determine reliability and acceptability of the decision-making of the AI.

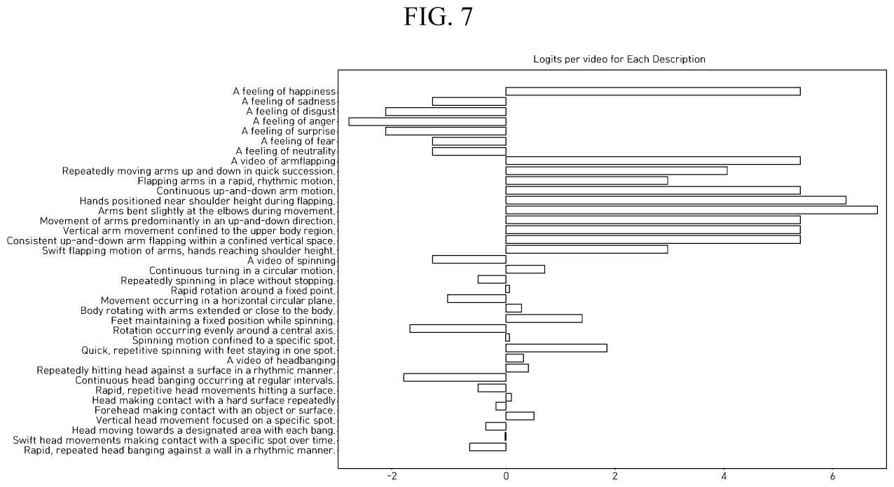

FIG. 7 illustrates an example of intermediate concept information according to an embodiment.

Referring to FIG. 7, a processor 110 (e.g., a composite information-integrated stereotyped action recognition inference framework) according to the embodiment may represent the similarity-related information-between the input video data and the list of composite description phrases-which is generated by the intermediate concept generation unit 118, as a graph.

As described above, the apparatus 100 for recognizing a stereotyped action according to the embodiment may provide not only the type of a child's stereotyped action included in each input video but also an interpretable output regarding the similarity to a list of composite (action) description phrases and an emotion associated with the action, and thus may provide assistance in action analysis and clinical decision making of ASD children.

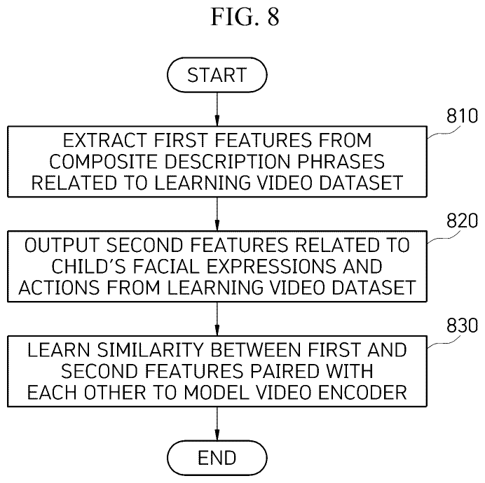

FIG. 8 is a flowchart showing a method of learning stereotyped action recognition according to an embodiment.

Referring to FIG. 8, in operation 810, the apparatus 100 for recognizing a stereotyped action may encode at least one composite description phrase related to a learning video dataset to extract first features.

In operation 820, the apparatus 100 for recognizing a stereotyped action may encode the learning video dataset through the video encoder 116 to output second features related to a user's facial expressions and actions included in a video. In operation 830, the apparatus 100 for recognizing a stereotyped action may learn the similarity between the first and second features that are paired with each other among the first features and the second features, to model the video encoder 116 such that the similarity between the first and second features that are paired with each other increases. Accordingly, the apparatus 100 for recognizing a stereotyped action may adjust the weight of the video encoder 116 such that the video encoder 116 may extract, from each piece of video data, features that are more similar to the composite (action) description phrases related to the actions and expressions included in each video.

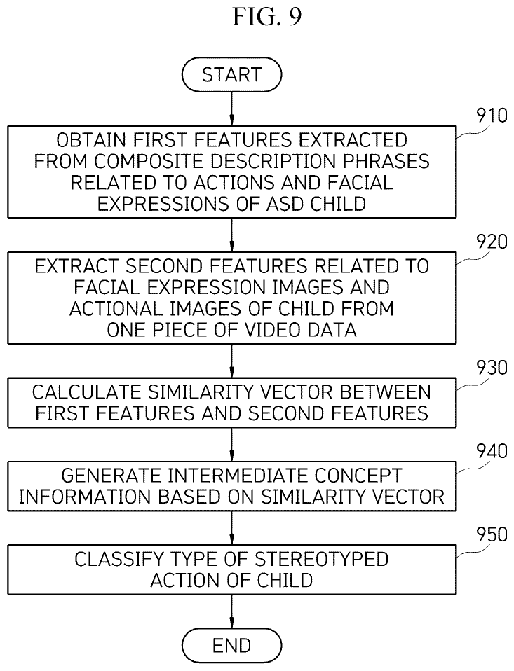

FIG. 9 is a flowchart showing a method of recognizing a stereotyped action according to an embodiment.

Referring to FIG. 9, in operation 910, the apparatus 100 for recognizing a stereotyped action may obtain first features of a list of composite description phrases describing stereotyped actions of an ASD child in relation to facial expressions, for example, from the memory 130.

In operation 920, the apparatus 100 for recognizing a stereotyped action may extract second features related to actional images and facial expression images of a diagnosis subject from one piece of input video data.

In operation 930, the apparatus 100 for recognizing a stereotyped action may calculate a similarity vector between the first features and the second features. For example, the apparatus 100 for recognizing a stereotyped action may calculate a similarity vector between the first features and the second features related to each composite description phrase in the list.

In operation 940, the apparatus 100 for recognizing a stereotyped action may generate intermediate concept information based on the similarity vector.

In operation 950, the apparatus 100 for recognizing a stereotyped action may classify (infer) the type of the action included in the one piece of video data based on the similarity between the first features and the second features.

The various embodiments of the disclosure and terminology used herein are not intended to limit the technical features of the disclosure to the specific embodiments, but rather should be understood to cover all modifications, equivalents, and alternatives falling within the spirit and scope of the invention. Like numbers refer to like elements throughout the description of the drawings. The singular forms preceded by “a” and “an” corresponding to an item are intended to include the plural forms as well unless the context clearly indicates otherwise. In the disclosure, a phrase such as “A or B”, “at least one of A and B”, “at least one of A or B”, “A, B or C”, “at least one of A, B and C”, or “at least one of A, B, or C” may include any one of the items listed together in the corresponding phrase, or any possible combination thereof. Terms such as “first”, “second”, etc., are used to distinguish one element from another and do not modify the elements in other aspects (e.g., importance or sequence). When one (e.g., a first) element is referred to as being “coupled” or “connected” to another (e.g., a second) element with or without the term “functionally” or “communicatively”, it means that the one element is connected to the other element directly (e.g., by wire), wirelessly, or via a third element.

As used herein, the term “module” may include units implemented in hardware, software, or firmware, and may be interchangeably used with terms such as “logic”, “logic block”, “component”, or “circuit.” The module may be an integrally formed component or a minimum unit or part of the integrally formed component that performs one or more functions. For example, according to an embodiment, the module may be implemented in the form of an application-specific integrated circuit (ASIC).

The various embodiments of the present disclosure may be realized by software (e.g., a program) including one or more instructions stored in a storage medium (e.g., the memory 130 in FIG. 1) (e.g., an internal memory or external memory) that may be read by a machine (e.g., an electronic device). For example, a processor (e.g., the processor 110 in FIG. 1) of the machine (e.g., the apparatus 100 for recognizing a stereotyped action) may invoke and execute at least one instruction among the stored one or more instructions from the storage medium. Accordingly, the machine operates to perform at least one function in accordance with the invoked at least one command. The one or more instructions may include code generated by a compiler or code executable by an interpreter. The machine-readable storage medium may be provided in the form of a non-transitory storage medium. Here, when a storage medium is referred to as “non-transitory”, it may be understood that the storage medium is tangible and does not include a signal (for example, electromagnetic waves), but rather that data is semi-permanently or temporarily stored in the storage medium.

According to an embodiment, the methods according to the various embodiments disclosed herein may be provided in a computer program product. The computer program product may be traded between a seller and a buyer as a product. The computer program product may be distributed in the form of a machine-readable storage medium (e.g., a compact disc read only memory (CD-ROM)) or may be distributed directly between two user devices (e.g., smartphones) through an application store (e.g., Play Store™), or online (e.g., downloaded or uploaded). In the case of online distribution, at least a portion of the computer program product may be stored at least semi-permanently or may be temporarily generated in a machine-readable storage medium, such as a memory of a server of a manufacturer, a server of an application store, or a relay server.

Components according to various embodiments of the disclosure may be implemented in the form of software or hardware, such as a digital signal processor (DSP), a field-programmable gate array (FPGA) or an ASIC, and may perform predetermined functions. The term “elements” is not limited to meaning software or hardware. Each of the elements may be stored in a storage medium capable of being addressed and configured to execute one or more processors. For example, the elements may include elements such as software elements, object-oriented software elements, class elements, and task elements, processes, functions, attributes, procedures, subroutines, segments of program code, drivers, firmware, microcode, circuits, data, databases, data structures, tables, arrays, and variables.

According to the various embodiments, each of the above-described elements (e.g., a module or a program) may include a singular entity or a plurality of entities. According to various embodiments, one or more of the above-described elements or operations may be omitted, or one or more other elements or operations may be added. Alternatively, or additionally, a plurality of elements (e.g., modules or programs) may be integrated into one element. In this case, the integrated element may perform one or more functions of each of the plurality of elements in a manner the same as or similar to that performed by the corresponding element of the plurality of components before the integration. According to various embodiments, operations performed by a module, program, or other elements may be executed sequentially, in parallel, repeatedly, or heuristically, or one or more of the operations may be executed in a different order, or omitted, or one or more other operations may be added.

According to various embodiments disclosed in this document, actions and facial expressions of video data can be analyzed to assist in the diagnosis of children with autism spectrum disorder. In addition, various effects that are directly or indirectly identified through this document may be provided.

What is claimed is:

1. An apparatus for recognizing a stereotyped action, comprising:
a memory storing a learning video dataset including a stereotyped action of a designated disabled child; and
a processor functionally connected to the memory,
wherein the processor comprises:
a text encoder configured to extract first features from a composite description phrase related to a facial expression and an action of the child included in the learning video dataset;
a video encoder configured to output second features related to a facial expression and an action of the child from the learning video dataset; and
a contrastive learning unit configured to learn a similarity between the first and second features forming a pair among the first features and the second features to model the video encoder.

2. The apparatus of claim 1, wherein the video encoder has a structure based on a 3D convolutional neural network (CNN) and a video transformer to utilize temporal information of time-series data.

3. The apparatus of claim 1, wherein the processor includes:
an emotion action recognition unit configured to output an emotion word corresponding to the facial expression;
an action description generation unit configured to generate a plurality of action description phrases for each stereotyped action; and
a linkage unit configured to combine at least one of the plurality of action description phrases with the emotion word to generate the composite description phrase.

4. The apparatus of claim 3, wherein the emotion action recognition unit is configured to:
extract an expression feature from a face region included in the learning video dataset, classify an emotion of the child based on the extracted expression feature using a designated categorial model; and
output the emotion word corresponding to the classified emotion.

5. The apparatus of claim 4, wherein the processor further includes a preprocessing unit configured to detect the face region from the learning video dataset and provide the detected face region to the video encoder and the emotion action recognition unit.

6. The apparatus of claim 3, wherein the action description generation unit generates the plurality of action description phrases corresponding to action label information of the stereotyped action using a large language model.

7. The apparatus of claim 3, wherein the linkage unit randomly selects a single action description phrase from among the plurality of action description phrases and combines the selected action description phrase with the emotion word to generate the composite description phrase.

8. The apparatus of claim 1, wherein the video dataset includes a plurality of pieces of video data each including a different stereotyped action, and
the contrastive learning unit adjusts a weight of the video encoder such that a similarity between first and second features forming the pair among the second features and the first features extracted from each of the plurality of pieces of video data is maximized and a similarity between first and second features forming different pairs is minimized.

9. The apparatus of claim 1, wherein the processor further includes an intermediate concept generation unit, and
the intermediate concept generation unit is configured to:
obtain a plurality of first features related to a list of composite description phrases regarding a plurality of stereotyped actions of the designated disabled child;
obtain a second feature related to a stereotyped action and a facial expression included in one piece of video data from the modeled video encoder, and
generate similarity-related information between the list of composite description phrases and the action and the facial expression in the one piece of video data.

10. The apparatus of claim 9, wherein the processor further includes a stereotyped action recognition unit, and
the stereotyped action recognition unit infers a type of the stereotyped action based on the similarity-based information and outputs an inference result.

11. An apparatus for recognizing a stereotyped action recognition device, comprising:
a memory storing first features of a list of composite description phrases describing a stereotyped action of a designated disabled child in relation to a facial expression; and
a processor functionally connected to the memory,
wherein the processor comprises:
a video encoder configured to extract second features related to an action and a facial expression of a subject to be diagnosed from one piece of video data; and
an action recognition unit configured to infer a type of the action included in the one piece of video data based on the similarity between the first features and the second features.

12. The apparatus of claim 11, wherein the processor further includes an intermediate concept generation unit configured to calculate a similarity between the first features and the second features and generate similarity-related information between the list of composite description phrases and the action and the facial expression in the one piece of video data.

13. The apparatus of claim 12, further comprising an output device,
wherein the processor organizes the similarity-related information in at least one visual format of a graph and a chart and outputs the organized similarity-related information in the at least one visual format through the output device.

14. The apparatus of claim 11, wherein the processor further includes a text encoder configured to extract the first features from the list of composite description phrases and store the extracted first features in the memory.

15. The apparatus of claim 11, wherein the processor further includes a text encoder and a contrastive learning unit,
the text encoder encodes a composite descriptive phrase describing each facial expression related to each stereotyped action in learning video data captured for the designated disabled child to extract first features,
the video encoder extracts second features related to each stereotyped action and the facial expression from the learning video data, and
the contrastive learning unit learns a similarity between the first and second features that are paired with each other among the first features and the second features extracted from the learning video data to model the video encoder.

16. A method of recognizing a stereotyped action, which is performed by at least one processor, comprising:
encoding at least one composite descriptive phrase related to a learning video dataset to extract first features;
encoding the learning video dataset using a video encoder to output second features related to a facial expression and an action of a subject to be diagnosed; and
learning a similarity between the first and second features that are paired with each other among the first features and the second features to model the video encoder such that the similarity between the first and second features paired with each other increases.

17. The method of claim 16, further comprising: before the extracting of the first features,
outputting an emotion word corresponding to the facial expression;
generating a plurality of action description phrases for each of the stereotyped actions using a large language model; and
combining at least one of the plurality of action description phrases with the emotion word and generating the composite description phrase.

18. The method of claim 17, wherein the generating of the composite description phrase includes:
randomly selecting a single action description phrase from among the plurality of action description phrases; and
combining the selected action description phrase with the emotion word to generate the composite description phrase.

19. The method of claim 17, further comprising:
obtaining a plurality of first features related to a list of composite description phrases regarding a plurality of stereotyped actions of the designated disabled child and obtaining a second feature related to a stereotyped action and a facial expression included in one piece of video data from the modeled video encoder;
generating similarity-related information between the list of composite description phrases and the action and the facial expression in the one piece of video data; and
outputting the similarity-related information.

20. The method of claim 19, wherein the outputting of the similarity-related information includes at least one of:
inferring a type of the stereotyped action based on the similarity-based information of the action and the facial expression in the one piece of video data, and outputting an inference result; and
visualizing and outputting the similarity-related information.
